(19) United States
(12) Patent Application Publication
(10) Pub. No.: US 2024/0393219 A1
(43) Pub. Date: Nov. 28, 2024

(54) DEVICE FOR MONITORING LIQUID, IN PARTICULAR FERMENTABLE LIQUID, SUCH AS WORT, IN PARTICULAR DURING THE FERMENTATION OF SAID LIQUID

(71) Applicant: My Bacchus, Saint Herblain (FR)

(72) Inventor: Alexandre ERMENAULT, SAINT HERBLAIN (FR)

(21) Appl. No.: 18/690,095

(22) PCT Filed: Sep. 9, 2022
(86) PCT No.: PCT/FR2022/051707

§ 371 Date: Mar. 7, 2024

(30) Foreign Application Priority Data
Sep. 10, 2021 (FR) 2109519

(51) Int. Cl. G01N 9/12

(52) U.S. Cl. CPC G01N 9/12

(57) ABSTRACT

A device for monitoring liquid includes an apparatus for measuring a parameter representative of the density of the liquid to be monitored. This measurement apparatus includes at least one floating structure, at least one member for determining the position of at least a part of the floating structure, and at least one transmitter for transmitting data. The device includes an enclosure accommodating the floating structure and a system for hanging the enclosure. The enclosure has a first end and extends from the first end in the direction of the opposite second end, forming a chamber delimited by a solid wall and a porous wall so as to form inside the chamber, in the manner of a diving bell, with a first space in which air is able to be trapped in the submerged state of the enclosure and a second space inside which the liquid to be monitored can circulate. The hanging system includes a suspension rod connected at one end to the enclosure and equipped at its opposite end with a hanging member for keeping the enclosure in the suspended state.

The present invention relates to a device for monitoring liquid, in particular fermentable liquid, such as must, in particular during the fermentation of said liquid.

It relates in particular to a device for monitoring liquid, in particular fermentable liquid, said device comprising at least one measurement apparatus for measuring a parameter representative of the specific gravity and/or the density of the liquid to be monitored, this measurement apparatus comprising at least one floating structure, at least one member for determining the position of at least a portion of the floating structure in order to make it possible to supply position data as a function, with said floating structure at least partially immersed in the liquid to be monitored, of at least the specific gravity and/or the density of the liquid in which the floating structure is at least partially immersed, and at least one transmitter capable of transmitting data as a function of the position data supplied by the at least one member for determining the position of at least a portion of the floating structure.

Many fruit or vegetable juices are subjected to fermentation, in particular for the production of beverages. Such is the case of grape juice, also called must, the fermentation of which makes it possible to produce wine. During this alcoholic fermentation, sugars in the juice are converted to alcohol. One of the means for monitoring this fermentation, which takes place in tanks, is to regularly take samples which are subjected to measurements, in particular specific gravity and/or density measurements. It is thus possible to track changes in the specific gravity and/or the density of the must tested. This specific gravity or this density changes over time, decreasing, reflecting an increase in the degree of alcohol in the liquid. When the specific gravity or the density has reached a predetermined value or no longer changes, fermentation is considered to be complete. However, such a procedure is tedious and unreliable. There are moreover devices, also known as hydrometers, for measuring the specific gravity and/or the density of a liquid in real time and continuously. Such is the case for example of the hydrometer described in patent US2014/0260607. The principle of such a hydrometer is to tilt as a function of the specific gravity of the liquid in which it floats and to measure said tilt in order to deduce therefrom the specific gravity or the density of the liquid. However, such a hydrometer requires an undisturbed environment for carrying out the measurements. Such systems have never been used for monitoring the fermentation of grape juice with a view to the production of red wine or white wine. This is because the presence of a cap formed by the marc during the fermentation of juice for the production of red wine prevents the hydrometer from floating freely. The presence of significant movements of the liquid resulting, during the fermentation of juice for the production of white wine, from gaseous discharges, falsifies the tilt of the hydrometer and consequently the measurements provided by said hydrometer.

One aim of the invention is to propose a device for monitoring liquid, in particular fermentable liquid, the design of which allows real-time and continuous monitoring of fermentation, even in liquids used for the production of alcoholic beverages.

To this end, the subject of the invention is a device for monitoring liquid, in particular fermentable liquid, said device comprising at least one measurement apparatus for measuring a parameter representative of the specific gravity and/or the density of the liquid to be monitored, this measurement apparatus comprising at least one floating structure, at least one member for determining the position of at least a portion of the floating structure in order to make it possible to supply position data as a function, with said floating structure at least partially immersed in the liquid to be monitored, of at least the specific gravity and/or the density of the liquid in which the floating structure is at least partially immersed, and at least one transmitter capable of transmitting data as a function of the position data supplied by the at least one member for determining the position of at least a portion of the floating structure, characterized in that the device comprises a casing for housing the floating structure and an attachment system for attaching the casing, in that said casing, intended to be immersed in the liquid to be monitored, has a first end referred to as closed and a second end and extends from the first end in the direction of the second end, forming a chamber delimited by a solid wall and a wall referred to as the porous wall provided with through openings so as to form, inside the chamber, in the manner of a diving bell, at least a first space referred to as the upper space, delimited at least by the solid wall and in which air can be trapped in the immersed state of the casing, and a second space referred to as the lower space, delimited by the porous wall and inside which the liquid to be monitored can circulate, and in that the attachment system comprises at least one rod referred to as the suspension rod, connected at one end to the casing and equipped at its opposite end with at least one attachment member to allow the casing to be held in the suspended state in a position in which the first end of the casing lies above the second end of the casing, and in that the floating structure of the measurement apparatus is a floating structure configured to float freely with free orientation inside the casing. The presence of a casing for housing the floating structure inside which the structure, surrounded by said casing, can take up several positions makes it possible to protect the floating structure from the environment. The production of the casing in the form of a chamber in the manner of a diving bell makes it possible, in the at least partially immersed state of the casing, to form, inside the casing, at the interface between the volume of trapped air and the body of circulating liquid, a surface referred to as the surface for free flotation of the floating structure. The floating structure may thus float freely inside the casing without being disturbed by the environment and may take up a position as a function of the specific gravity of the liquid in which it floats. A “floating structure configured to float freely with free orientation inside the casing” means that the floating structure of the measurement apparatus does not need to be attached to the casing in which it floats, or to any element whatsoever, and it is not forced to move, in particular in terms of orientation, by any actuator, such as an electrical or electromagnetic device that is internal and/or external to said floating structure. The measurement apparatus is a hydrometer. The casing makes it possible to provide a volume dimensioned in such a way that, inside said volume, the floating structure can float and take up a flotation position as a function of the specific gravity of the liquid in which it floats, when the casing is at least partially immersed in said liquid. The end of the casing that is closed in an airtight and watertight manner makes it possible, in collaboration with the solid wall, to reliably have a sealed volume capable of trapping air. The presence of the system for attaching the casing and its design make it possible to ensure, when the casing is at least partially immersed in the liquid to be monitored, that the casing is positioned such that the first space lies above the second space such that the formation of a flotation surface at the interface between the volume of trapped air and the body of circulating liquid is guaranteed.

According to one embodiment of the invention, the device comprises at least one filter element that may be placed around the porous wall of the casing and the filter element is a perforated flexible plate, in which the perforations have, preferably, a diameter of between 2 and 5 mm, even more preferably close to 3.15 mm. The presence of a filter element of strainer type around the porous wall of the casing makes it possible to filter the largest particles contained in the liquid to be monitored and thus prevent these particles accumulating in the casing. Such an accumulation could obstruct the openings in the porous wall and prevent circulation of the fluid through the casing.

The perforated flexible plate may be in the form of a mesh plate.

According to one embodiment of the invention, the through openings in the porous wall of the casing are circular openings and the through openings in the porous wall of the casing have a diameter at least equal to 0.5 mm and less than 30 mm, preferably between 0.5 mm and 15 mm, preferably close to 10 mm. The presence of circular openings allows better circulation of the liquid inside the casing.

The diameters of the circular holes in the porous wall of the casing are determined to ensure homogeneity in the measurement and to prevent any disruption of the tilting of the floating structure of the measurement apparatus, in particular as a result of contact between the floating structure and the wall of the casing as it tilts as a function of the specific gravity of the contents of the casing. The presence of the circular openings and their dimension make it possible, under the effect of the fermentation, to have fine bubbles which tend to center the measurement apparatus. The circularity of the openings tends to “cut” the bubbles. A diameter of the openings of less than 0.5 mm does not make it possible to obtain homogeneity in the measurement. A diameter of the through openings in the porous wall of greater than 30 mm generates too much disturbance, making the measurement apparatus unsuitable for the measurement.

According to one embodiment of the invention, the casing is cylindrical over at least a portion of its length considered between its first end and its second end, the floating structure of the measurement apparatus is a structure in the form of a watertight elongate hollow body, and the diameter of the cylindrical portion of the casing is at least equal to 1.3 times the length of the hollow body of the floating structure. This arrangement allows the floating structure to orient itself, specifically to tilt, as a function of the specific gravity of the liquid without the risk of friction with the casing. Preferably, the cylindrical portion of the casing has a diameter at least equal to 70 mm, preferably between 100 mm and 160 mm.

Preferably, the casing has an inner length considered between its first end and its second end at least equal to 120 mm, preferably between 130 mm and 230 mm.

According to one embodiment of the invention, the inner length of the casing considered between the first end of the casing and the interface between the first upper space and the second lower space is at least equal to twice the length of the hollow body of the floating structure.

According to one embodiment of the invention, the inner length of the casing considered between the second end of the casing and the interface between the first upper space and the second lower space is at least equal to 1.5 times the length of the hollow body of the floating structure. These arrangements make it possible to ensure reliable measurement by the measurement apparatus, the floating structure of which is configured to float freely with free orientation.

According to one embodiment of the invention, at least some of the through openings in the porous wall of the casing are arranged in an area where the porous wall is joined to the second end of the casing. Again, this arrangement makes it possible to facilitate the flow of the liquid and to prevent the accumulation of particles within the casing.

According to one embodiment of the invention, the second end of the casing is delimited by at least one piece, mounted movably relative to the porous wall of the casing so that said second end can go from a closed position to an open position or vice versa. The second end of the casing is preferably also a closed end. The production of the second end of the casing in the form of a movable end makes it possible to open the casing via said second end. This makes it possible to easily place at least the floating structure of the measurement apparatus inside the casing.

According to one embodiment of the invention, the second end of the casing is a conical end, said second end being delimited by a cone connected via its base forming a solid surface, preferably flat, to the porous wall of the casing, said cone preferably being removably fastened to the porous wall of the casing. The conical shape makes it possible to facilitate the penetration of the casing into the liquid to be monitored.

The fact that the cone is removably fastened to the porous wall of the casing makes it possible to easily place at least the floating structure of the measurement apparatus inside the casing.

According to one embodiment of the invention, the or at least one of the attachment members has the shape of a hook. This hook facilitates the attachment of the device at the upper edge of a fermentation tank.

According to one embodiment of the invention, the or at least one of the attachment members has the shape of a bar extending transversely to the rod. This design makes it possible to position the bar horizontally on the opening of a tank so as to simply hold the device in the suspended state.

According to one embodiment of the invention, the device comprises a data receiver and a transmission relay, such as an antenna, arranged between the transmitter and the receiver, at least a portion of the relay being positioned inside the rod, which is a hollow rod. The presence of a transmission relay makes it possible to ensure good transmission of the measured data in all circumstances and whatever the nature of the liquid medium in which the device is placed.

According to one embodiment of the invention, the floating structure of the measurement apparatus is a structure in the form of a watertight elongate hollow body, this floating structure having a center of gravity and a center of buoyancy which do not coincide so as to take up a position as a function of the specific gravity of the liquid in which the structure floats, the or at least one of the members for determining the position of at least a portion of the floating structure capable of supplying position data is a member for determining the tilt of at least a portion of the floating structure with respect to the vertical, such as an accelerometer, and said measurement apparatus comprises a power supply source, such as an accumulator. The advantage of such a design lies in the ease of replacement of the measurement apparatus in the event of failure. All that is required is to open the casing and remove the floating structure and replace it. The floating structure is in fact arranged freely inside the casing without being anchored to the casing.

According to one embodiment of the invention, the measurement apparatus does not have means for control in orientation by magnetization or electromagnetism of the floating structure.

According to one embodiment of the invention, the device comprises at least one electronic and/or computer module for processing the position measurement data so as to determine the specific gravity and/or the density of the liquid from said data.

Another subject of the invention is a method for monitoring liquid using a device for monitoring liquid, characterized in that the device for monitoring liquid being of the aforementioned type, the method comprises, with the floating structure in position in the casing, a step of placing the casing in the liquid to be monitored in a position in which the first end of the casing lies above the second end of the casing and at least the porous wall of the casing is immersed in the liquid, and a step of attaching the device in said position.

### BRIEF DESCRIPTION OF THE DRAWINGS

A clear understanding of the invention will be obtained on reading the following description of exemplary embodiments, with reference to the appended drawings in which:

FIG. 1 is a basic schematic view of a device according to the invention in the configuration for use in a liquid fermentation tank for the production of red wine;

FIG. 2 is a schematic view of a device according to the invention with a hook-type attachment member;

FIG. 3 is a schematic view of a device according to the invention with a bar-type attachment member;

FIG. 4 is a partial view of a device according to the invention;

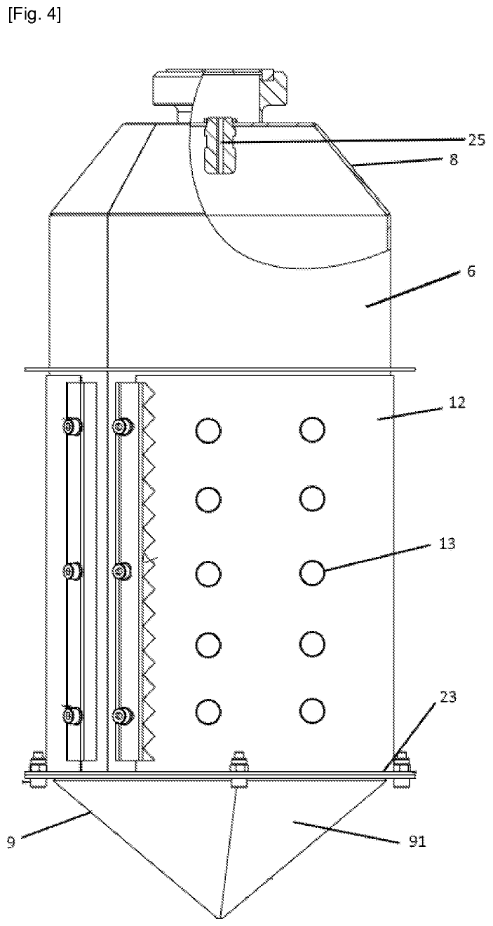

FIG. 5 is an end-on view of the conical second end of the casing;

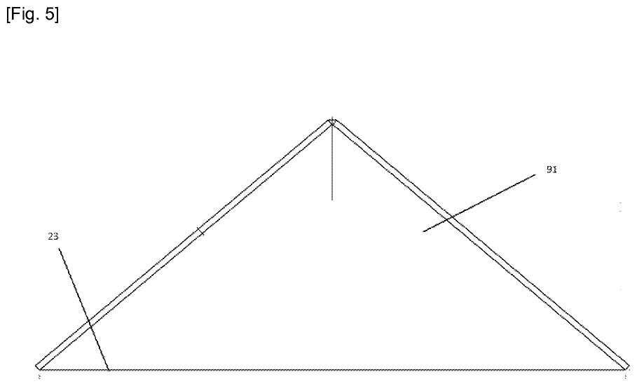

FIG. 6 is a schematic view of a measurement apparatus;

FIG. 7 is a partially transparent partial view of a device according to the invention;

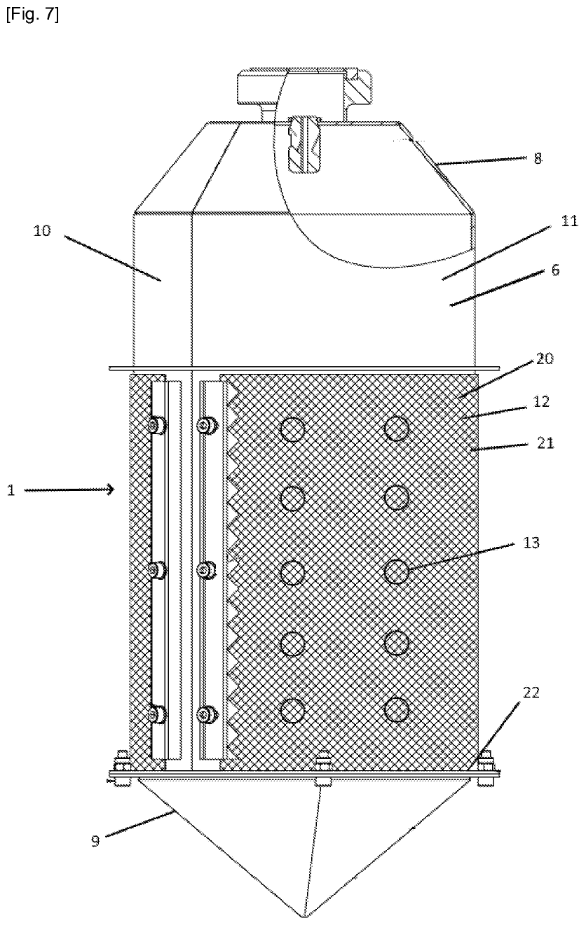

FIG. 8 is a partially transparent partial view of a device according to the invention, in situ.

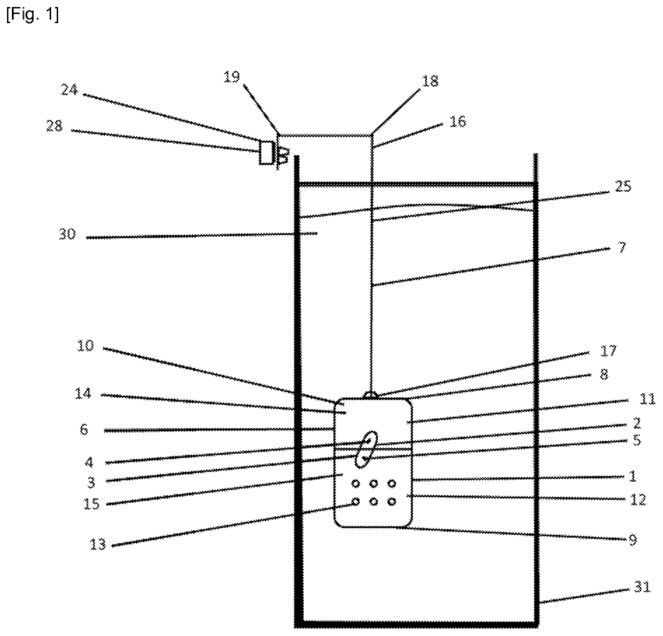

As mentioned above, the subject of the invention is a device 1 for monitoring liquid, in particular fermentable liquid, as in the example shown in FIG. 1, in which the liquid to be monitored is a grape must for the production of red wine.

The liquid 30 is therefore contained in a fermentation tank 31.

The device 1 that is the subject of the invention is intended to be at least partially immersed in the liquid present under the cap formed by the marc.

This device 1 is intended to monitor the fermentation of the liquid by measuring a parameter representative of the specific gravity and/or the density of this liquid. This specific gravity and/or this density decreases over time, following the conversion of the sugars to alcohol. The end of alcoholic fermentation corresponds either to a stabilization of the specific gravity and/or the density of the liquid or to a predetermined value of this specific gravity and/or this density.

The device 1 that is the subject of the invention allows real-time and continuous measurement of a parameter representative of this specific gravity and/or this density.

The device 1 therefore comprises a measurement apparatus comprising at least one floating structure 3, at least one member 4 for determining the position of at least a portion of the floating structure 3 in order to make it possible to supply position data as a function, with said floating structure 3 at least partially immersed in the liquid 30 to be monitored, of at least the specific gravity and/or the density of the liquid 30 in which the floating structure 3 is at least partially immersed, and at least one transmitter 5 capable of transmitting data as a function of the position data supplied by at least the member 4 for determining the position of at least a portion of the floating structure 3.

The floating structure 3 or at least a portion of the floating structure 3 is sensitive to the specific gravity and/or the density of the liquid 30 in which the floating structure 3 is able to float in order, when it is at least partially immersed in a liquid, to take up a position as a function of the specific gravity of the liquid in which the floating structure 3 floats.

The floating structure 3 or at least a portion of the floating structure 3 performs the function of densimeter and is therefore configured so as, when it is at least partially immersed in a liquid, to take up a position as a function of the specific gravity of the liquid in which the floating structure 3 floats.

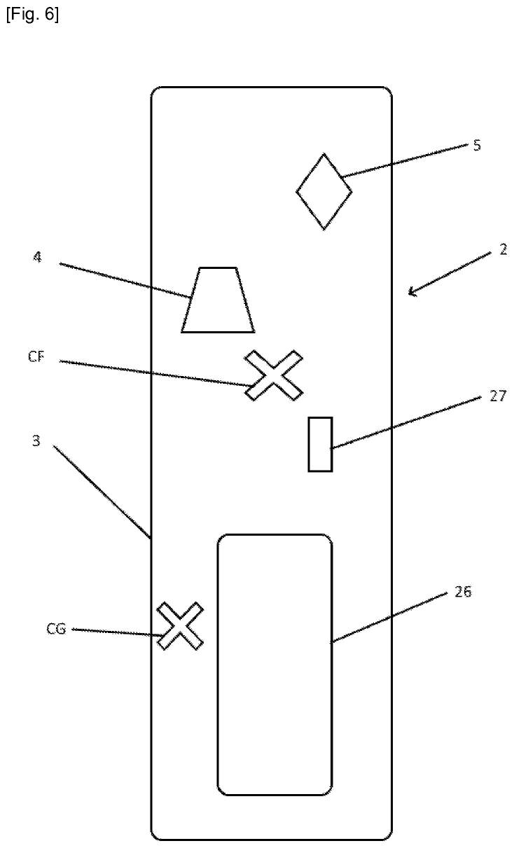

Details of such a measurement apparatus are shown in FIG. 6. In this example, the floating structure 3 of the measurement apparatus is a structure in the form of a watertight elongate hollow body. This floating structure 3 has a center of gravity (CG) and a center of buoyancy, also referred to as bottom center (CF), which do not coincide, so as to take up a position as a function of the specific gravity of the liquid in which the floating structure 3 floats.

In the example shown, the hollow body of the floating structure 3 is cylindrical in shape. The floating structure 3 tends to tilt as a function of the thrust forces to which it is subjected when it floats. These thrust forces vary as a function of the density and/or the specific gravity of the liquid, based on the Archimedes principle.

The floating structure 3 of the measurement apparatus 2 is a floating structure 3 configured to float freely with free orientation inside the casing 6. In other words, the measurement apparatus 2 does not have means for control in orientation by magnetization or electromagnetism of the floating structure 3. The floating structure which floats freely is therefore autonomous and does not need anchoring or an actuator that would force it to orient itself in a predetermined manner. Under these conditions, the measurement apparatus 2 is a hydrometer.

The member 4 for determining the position of the floating structure 3 or at least a portion of the floating structure 3 is a member for determining the tilt of at least a portion of the structure with respect to the vertical, in this case of the entire structure with respect to the vertical.

This determining member 4 is in this case produced in the form of an accelerometer.

This member 4 for determining the position of the floating structure 3 is housed inside the floating structure 3, but it could be fastened to the floating structure 3 without departing from the scope of the invention.

The measurement apparatus 2 further comprises a power supply source 26, such as an accumulator. In the example shown, this accumulator is a cell placed in the watertight floating structure 3.

In the example shown, the measurement apparatus further comprises a temperature sensor 27, able to supply temperature data concerning the liquid surrounding the measurement apparatus.

Again, this temperature sensor is arranged on or in the floating structure 3.

Details of the measurement apparatus 2 will not be set out below, since it is well known to those skilled in the art in question, as can be seen for example in patent U.S. Pat. No. 9,234,828. Naturally, any other measurement apparatus performing the function of densimeter incorporating a floating structure 3 floating freely with free orientation could be used, without departing from the scope of the invention.

The measurement apparatus 2 further comprises a transmitter 5 capable of transmitting data, as a function of the position data supplied by the member 4 for determining the position of at least a portion of the floating structure 3. This transmitter 5 may be integrated into the floating structure or carried by the floating structure 3. The data transmitted may be raw or processed data.

In the examples shown, the device comprises a data receiver 24 and a transmission relay 25, such as an antenna, arranged between the transmitter 5 and the receiver 24.

The data is transmitted partly via radio link, for example via UHF radio link such as Bluetooth.

Thus, the data from the member 4 for determining the position of at least a portion of the floating structure 3 are transmitted by the transmitter 5 to a remote receiver 24 which is generally arranged outside the liquid 30 to be monitored.

The device 1 further comprises at least one electronic and/or computer module 28 for processing the position data, so as to determine the specific gravity and/or the density of the liquid from said data.

This electronic module 28 is a computer and/or electronic system that comprises at least a processor, a data storage memory and a program executable by the processor.

This electronic module 28 may incorporate a display device. This electronic module 28 may also be placed in communication with a remote terminal such as a computer, a cellphone or the like, on which the processed data may be displayed.

The device 1 comprises a casing 6 for housing the floating structure 3, inside which the floating structure 3 surrounded by said casing 6 is free to move, and an attachment system 7 for attaching the casing 6.

In the examples shown, the electronic module 28 described above is carried by the attachment system 7 as is the receiver 24, which may be integrated into the electronic and/or computer module 28.

The casing 6 intended to be immersed in the liquid 30 to be monitored has a first end 8 which is closed in a watertight and airtight manner, and a second end 9 which is preferably also closed in a watertight manner.

In this instance, the casing 6 is a casing of generally cylindrical shape with two ends of conical shape to give the casing 6 a cylindro-conical shape.

The casing 6 therefore extends between these two ends from the first end 8 in the direction of the second end 9, forming a chamber 10 delimited by a solid wall 11 and a wall referred to as the porous wall 12 provided with through openings 13 so as to form, inside the chamber 10, in the manner of a diving bell, at least a first space 14 referred to as the upper space, delimited at least by the solid wall 11 and in which air can be trapped in the immersed state of the casing 6, and a second space 15 referred to as the lower space, delimited by the porous wall 12 and inside which the liquid to be monitored can circulate.

Thus, a surface referred to as the free surface at which the floating structure 3 may float freely is formed in the casing 6, at the interface between the area in which air is trapped and the area in which the liquid circulates through the casing. This interface is shown in FIG. 1.

Thus, in this interface area, the member 4 for determining the position of at least a portion of the floating structure 3 determines the position, specifically the tilt, of the floating structure 3 and sends these position data via the transmitter 5 and in this case the transmission relay 25 to the receiver 24.

To allow the casing 6 to be immersed to the required level, that is to say to a level sufficient to trap a mass of air in the casing 6 and to hold it in position, an attachment system 7 for attaching the casing 6 is required. This attachment system 7 comprises a rod 16 referred to as the suspension rod connected at one end 17 to the casing 6 and equipped at its opposite end 18 with at least one attachment member 19 to allow the casing 6 to be held in the suspended state in a position in which the first end 8 of the casing 6 lies above the second end 9 of the casing 6. This rod 16 extends predominantly outside the casing 6.

In the examples shown, the rod 16 is a hollow rod and at least a portion of the transmission relay 25, in this case the antenna, is positioned inside the rod 16. The antenna therefore extends from the casing, inside the rod, until it protrudes on the outside of the end of the rod provided with the attachment member 19.

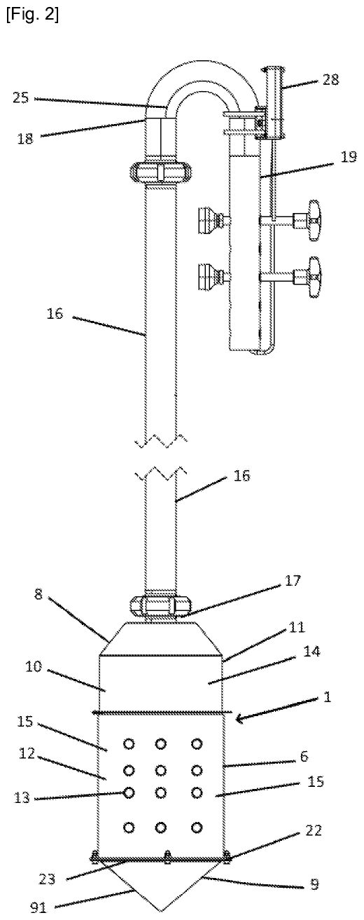

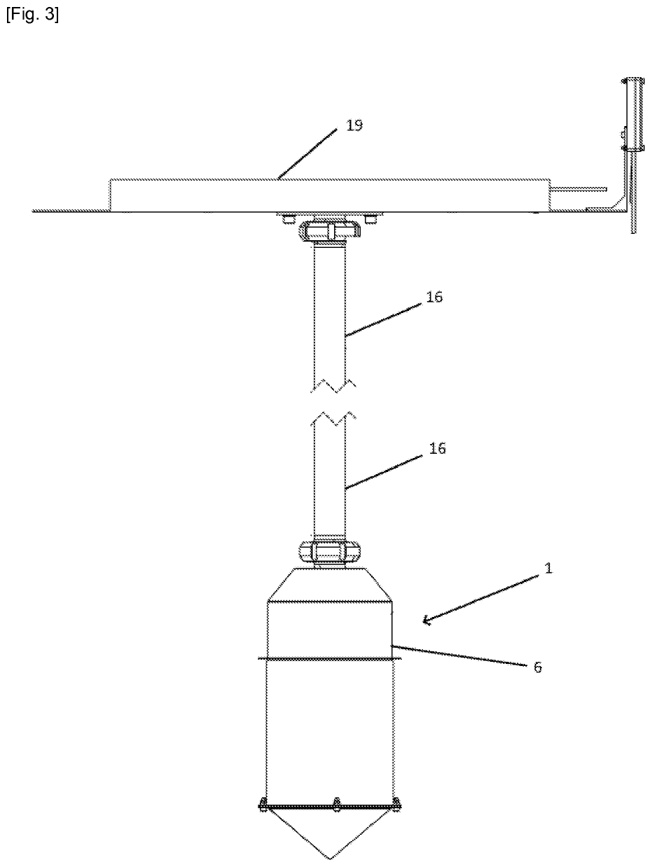

The rod is connected to the casing at the first end 8 of the casing 6. Thus, in the example shown in FIGS. 2 and 3, the first end of the casing is frustoconical in shape and is connected via its large base to the solid wall of the casing.

The rod is arranged at the center of the small base of the truncated cone and extends parallel to the longitudinal axis of the casing, which in this case corresponds to the median longitudinal axis of the cylinder. This rod is equipped with an attachment member 19.

In the example shown in FIG. 2, the attachment member 19 has the shape of a hook. This hook forms a U-shaped curved portion at the end of the rod, this U being open toward the casing. One arm of the U and the rod are coaxial. The other arm of the U is provided with clamping screws which form, with the rod 16, the clamping jaws of a vise between which a wall of the fermentation tank containing liquid to be monitored may be inserted.

Thus, the rod runs vertically inside the tank, while the attachment member 19 extends partially outside the tank, being arranged straddling an edge of the tank.

The casing 6 is thus held firmly in the position in which the first end 8 of the casing lies above the second end 9 of the casing 6.

The rod 16 may be of adjustable length. The same may apply to the attachment member 19 in order to vary the level of immersion of the casing 6.

FIG. 3 shows an attachment system 7, with an attachment member 19 which has the shape of a bar extending transversely to the rod 16. Thus, the bar may be positioned horizontally on the surface of a tank open at the top. This arrangement again allows the casing 6 to be held securely in the desired position.

To complete the device 1, the latter generally comprises at least one filter element 20 that may be placed around the porous wall 12 of the casing 6. In the examples shown, this filter element 20 is a perforated flexible plate, in particular a mesh plate, in which the perforations 21 or meshes have a diameter of between 2 and 5 mm, preferably close to 3.15 mm.

In the example shown, the filter element 20 is provided, at the two opposite edges of the plate, with a clamping strip. This clamping strip is itself secured to the casing 6.

This filter element 20 is prevented from moving axially along an axis parallel to the longitudinal axis of the casing 6 by two collars, one of which, referred to as the upper collar, is arranged in the transition area between the solid and porous walls of the casing 6, while the other, referred to as the lower collar, is arranged in the connection area where the porous wall of the casing is connected to the second end 9 of the casing 6.

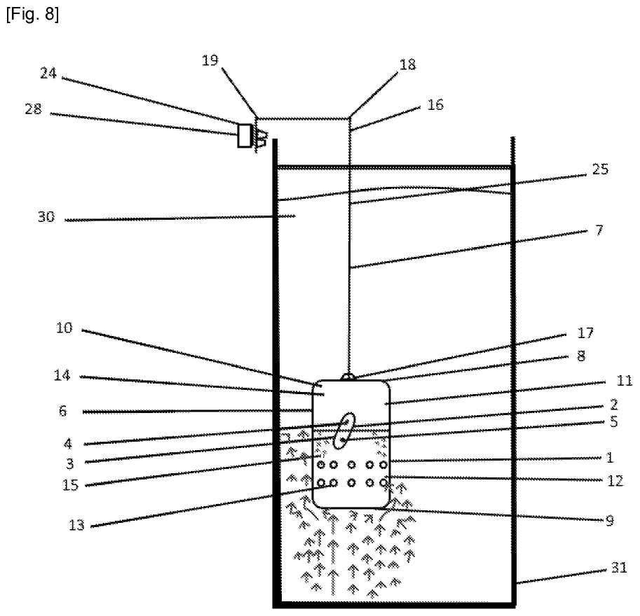

Note that the through openings 13 in the porous wall 12 of the casing 6 are circular openings 13. These through openings 13 in the porous wall 12 of the casing 6 have a diameter at least equal to 0.5 mm and less than 30 mm, preferably between 0.5 and 15 mm, preferably close to 10 mm. At least some of the through openings 13 in the porous wall 12 of the casing 6 are arranged in an area 22 where the porous wall 12 is joined to the second end 9 of the casing 6, in order to prevent any accumulation of particles there. All of the features of the openings described above tend to keep the floating structure of the measurement apparatus away from the wall of the casing as shown in FIG. 8.

The second end 9 of the casing 6 is, for its part, delimited by at least one piece, mounted movably relative to the porous wall 12 of the casing 6 so that said second end 9 can go from a closed position to an open position or vice versa.

In the examples shown, the second end 9 of the casing 6 is a conical end. This second end 9 is delimited by a cone 91 connected, via its base 23 forming a flat surface, to the porous wall 12 of the casing 6. Such a flat bottom of the casing makes it possible to prevent the accumulation of particles in the casing 6. The cone 91 is removably fastened to the porous wall 12 of the casing 6.

With the second end 9 of the casing 6 in the open position, it is possible to insert the floating structure 3 into the casing 6. The conical shape of this second end 9 helps the casing 6 penetrate the liquid. Similarly, the conical shape of the first end 8 of the casing 6 helps the device 1 emerge from the liquid 30. In regard to the fact that the floating structure 3 of the measurement apparatus 2 floats freely with free orientation inside the casing 6 and that it tends to tilt as a function of the density and/or the specific gravity of the liquid contained in the casing 6 on the basis of the Archimedes principle, it is imperative that the relative dimensioning of the casing and of the floating structure allows such movement without the casing interfering with the movements of the floating structure. For this purpose, it is important that the floating structure remain away from the wall serving to delimit the casing and that it can tilt to a greater or lesser degree without touching the casing 6. The casing 6 is cylindrical over at least a portion of its length considered between its first end 8 and its second end 9. Ideally, the diameter of the cylindrical portion of the casing 6 is at least equal to 1.3 times the length of the hollow body of the floating structure 3. Likewise, the inner length of the casing 6 considered between the first end 8 of the casing 6 and the interface between the first upper space 14 and the second lower space 15 is at least equal to twice the length of the hollow body of the floating structure 3 and the inner length of the casing 6 considered between the second end 9 of the casing 6 and the interface between the first upper space 14 and the second lower space 15 is at least equal to 1.5 times the length of the hollow body of the floating structure 3.

In the examples shown, the casing is made of metal. Generally, the casing has an inner length considered between its first end and its second end at least equal to 120 mm, preferably between 130 mm and 230 mm. The cylindrical portion of the casing has a diameter at least equal to 70 mm, preferably between 100 mm and 160 mm. In the example shown, the cylindrical portion of the casing has a height considered along the longitudinal axis of the cylinder, that is to say a length of 200 mm, and a diameter of 153 mm.

In practice, such a device 1 works as follows: it is assumed that the first end 8 of the casing 6, from which the antenna constituting the transmission relay 25 and the rod 16 of the attachment system 7 extend, is closed in an airtight and watertight manner. It is also assumed that the floating structure 3 has been inserted into the casing 6 via the second end 9 of the casing 6.

A first possibility consists in placing this casing 6 in the tank at the desired level, securing the casing 6 to the tank by means of the attachment system 7 and in particular by means of the attachment member 19, then filling the tank with liquid 30 to be monitored.

The position data supplied by the position determining member 4 are sent by the transmitter 5 via the transmission relay 25 to the receiver 24 and to the electronic and/or computer module 28 for processing of the data, which are secured to the attachment member 19 of the attachment system 7. The data may, after processing, and in particular conversion of the position data into data corresponding to the density and/or the specific gravity of the liquid, for example using suitable correspondence tables and optionally also taking into account the temperature, be displayed on the electronic and/or computer module or on a remote terminal.

The second possibility of implementation consists in inserting the casing 6 of the device 1 into a tank 31 already filled with liquid 30. Once the casing is placed in position in the tank and secured by its attachment system 7 to the tank, the measurement is performed in a manner identical to what has been described above.

In all cases, the final position of the casing 6 inside the tank must be such that air is trapped inside the casing 6.

1. A device for monitoring liquid, including fermentable liquids, said device comprising:
at least one measurement apparatus for measuring a parameter representative of the specific gravity and/or the density of the liquid to be monitored, this measurement apparatus comprising at least one floating structure, at least one member for determining the position of at least a portion of the floating structure in order to make it possible to supply position data as a function, with said floating structure at least partially immersed in the liquid to be monitored, of at least the specific gravity and/or the density of the liquid in which the floating structure is at least partially immersed, and at least one transmitter capable of transmitting data as a function of the position data supplied by the at least one member for determining the position of at least a portion of the floating structure,
wherein the device comprises a casing for housing the floating structure and an attachment system for attaching the casing, in that said casing, intended to be immersed in the liquid to be monitored, has a first end referred to as closed and a second end and extends from the first end in the direction of the second end, forming a chamber delimited by a solid wall and a wall referred to as the porous wall provided with through openings so as to form, inside the chamber, in the manner of a diving bell, at least a first space referred to as the upper space, delimited at least by the solid wall and in which air can be trapped in the immersed state of the casing, and a second space referred to as the lower space, delimited by the porous wall and inside which the liquid to be monitored can circulate, and in that the attachment system comprises at least one rod referred to as the suspension rod, connected at one end to the casing and equipped at its opposite end with at least one attachment member to allow the casing to be held in the suspended state in a position in which the first end of the casing lies above the second end of the casing, and in that the floating structure of the measurement apparatus is a floating structure configured to float freely with free orientation inside the casing.

2. The device for monitoring liquid as claimed in claim 1, wherein the device comprises at least one filter element that may be placed around the porous wall of the casing and in that the filter element is a perforated flexible plate, in which the perforations have a diameter of between 2 and 5 mm.

3. The device for monitoring liquid as claimed in claim 1, wherein the through openings in the porous wall of the casing are circular openings and in that the through openings in the porous wall of the casing have a diameter at least equal to 0.5 mm and less than 30 mm.

4. The device for monitoring liquid as claimed in claim 1, wherein the casing is cylindrical over at least a portion of its length considered between its first end and its second end, in that the floating structure of the measurement apparatus is a structure in the form of a watertight elongate hollow body, and in that the diameter of the cylindrical portion of the casing is at least equal to 1.3 times the length of the hollow body of the floating structure.

5. The device for monitoring liquid as claimed in claim 4, wherein the inner length of the casing considered between the first end of the casing and the interface between the first upper space and the second lower space is at least equal to twice the length of the hollow body of the floating structure.

6. The device for monitoring liquid as claimed in claim 4, wherein the inner length of the casing considered between the second end of the casing and the interface between the first upper space and the second lower space is at least equal to 1.5 times the length of the hollow body of the floating structure.

7. The device for monitoring liquid as claimed in claim 1, wherein at least some of the through openings in the porous wall of the casing are arranged in an area where the porous wall is joined to the second end of the casing.

8. The device for monitoring liquid as claimed in claim 1, wherein the second end of the casing is delimited by at least one piece, mounted movably relative to the porous wall of the casing so that said second end can go from a closed position to an open position or vice versa.

9. The device for monitoring liquid as claimed in claim 1, wherein the second end of the casing is a conical end, said second end being delimited by a cone connected via its base forming a solid surface to the porous wall of the casing.

10. The device for monitoring liquid as claimed in claim 1, wherein the or at least one of the attachment members has the shape of a hook.

11. The device for monitoring liquid as claimed in claim 1, wherein the or at least one of the attachment members has the shape of a bar extending transversely to the rod.

12. The device for monitoring liquid as claimed in claim 1, wherein the device comprises a data receiver and a transmission relay, including antennas, arranged between the transmitter and the receiver, at least a portion of the relay being positioned inside the rod, which is a hollow rod.

13. The device for monitoring liquid as claimed in claim 1, wherein the floating structure of the measurement apparatus is a structure in the form of a watertight elongate hollow body, this floating structure having a center of gravity and a center of buoyancy which do not coincide so as to take up a position as a function of the specific gravity of the liquid in which the structure floats, in that the or at least one of the members for determining the position of at least a portion of the floating structure capable of supplying position data is a member for determining the tilt of at least a portion of the floating structure with respect to the vertical, such as an accelerometer, and in that said measurement apparatus comprises a power supply source.

14. The device for monitoring liquid as claimed in claim 1, wherein the measurement apparatus does not have means for control in orientation by magnetization or electromagnetism of the floating structure.

15. The device for monitoring liquid as claimed in claim 1, wherein the device comprises at least one electronic and/or computer module for processing the position data so as to determine the specific gravity and/or the density of the liquid from said data.
